(19) United States
(12) Patent Application Publication
(10) Pub. No.: US 2013/0060030 A1
(43) Pub. Date: Mar. 7, 2013

(54) PROCESS FOR THE PREPARATION OF HIGHLY PURE CRYSTALLINE IMATINIB BASE

(71) Applicants: Amalia Kompella, Hyderabad (IN); Kali Satya Bhujanga Rao Adibhatla, Hyderabad (IN); Sreenivas Rachakonda, Hyderabad (IN); Venkaiah Chowdary Nannapeneni, Hyderabad (IN)

(72) Inventors: Amalia Kompella, Hyderabad (IN); Kali Satya Bhujanga Rao Adibhatla, Hyderabad (IN); Sreenivas Rachakonda, Hyderabad (IN); Venkaiah Chowdary Nannapeneni, Hyderabad (IN)

(73) Assignee: Natco Pharma Limited, Hyderabad (IN)

(21) Appl. No.: 13/634,725

(22) PCT Filed: Mar. 15, 2010
(86) PCT No.: PCT/IN10/00152

§ 371 Date: Nov. 20, 2012

(51) Int. Cl. C07D 403/14

(52) U.S. Cl. 544/295

(57) ABSTRACT

A process for the preparation of highly pure crystalline imatinib base form- N of Formula (I) is disclosed. Imatinib base of this invention is suitable for conversion to pharmaceutically acceptable salts.

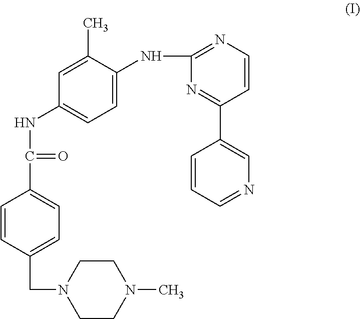 [SMILES: Cc1cc(NC(=O)c2ccc(CN3CCN(C)CC3)cc2)ccc1Nc1nccc(-c2cccnc2)n1]

### FIELD OF THE INVENTION

The present invention relates to the process for the preparation of crystalline Imatinib base of formula (I) and its solid state properties.

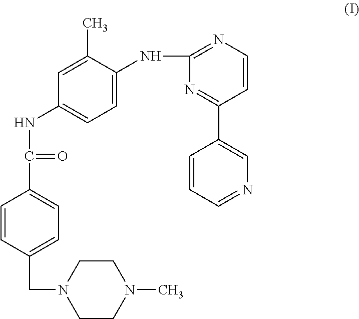 [SMILES: Cc1cc(NC(=O)c2ccc(CN3CCN(C)CC3)cc2)ccc1Nc1nccc(-c2cccnc2)n1]

### BACKGROUND OF THE INVENTION

Imatinib mesylate which is the methane sulfonate salt of N-{5-[4-(4-methylpiperazino-methyl)-benzoylamido]-2-methylphenyl}-4-(3-pyridyl) 2-pyrimidine-amine having the Formula I (a) I is approved under the trademark “Gleevec®” by the US Food and Drug Administration for the treatment of Chronic Myelogenous Leukemia before and after the failure of interferon alpha. It has also been approved for the treatment of patients with kit (CD117) positive unresectable and/or metastatic malignant Gastro Intestinal Stromal Tumors (GISTs) and also approved for the treatment of pediatric patients with philadelphia chromosome positive (Ph+) Chronic Myeloid Leukemia (CML) in chronic phase.

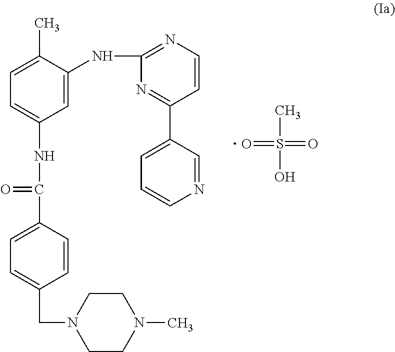

The preparation of N-{5-[4-(4-methylpiperazino-methyl)-benzoylamido]-2-methylphenyl}-4- (3-pyridyl) 2-pyrimidine-amine (Imatinib) of Formula (I) and the use thereof especially as an antitumour agent is described in EP0564 409, (Ciba-Geigy corp.) which was published on 6 Oct. 1993 and in U.S. 55211584 (Assignee: Ciba-Geigy corp; Title: Pyrimidine derivatives and process for the preparation there of) which was published on 28 May 1996 and in equivalent applications in numerous other countries. However, the solid state properties of imatinib base are not discussed here.

The preparation of Imatinib mesylate I(a) and the use thereof especially as an antitumour agent is described in WO99/03854, (Assignee: Novartis). This application describes two polymorphic forms of imatinib mesylate, the α-form and the β-form, WO 2005/075454 describes acid addition salts imatinib such as tartrate, citrate, malate, fumarate, etc., which are prepared by treatment of imatinib base with the corresponding acid.

In EP 0564409 and in its equivalent U.S. 55211584 the preparation of imatinib base having a melting point of 211-213° C., is described in example 21 (Scheme-1)

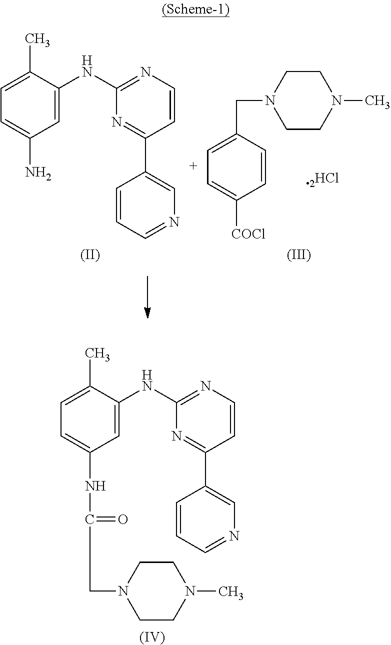

In this process disclosed in this patent a solution of N-(5-amino-2-methylphenyl)-4-(3-pyridyl)-2-pyrimidineamine hydrochloride of the formula (II) and 4-(4-methyl-piperazinomethyl)benzoyl chloride of the formula (III) taken in pyridine are stirred under nitrogen at room temperature for 23 hours. The resulting reaction mixture is concentrated under high vacuum; water is added and the mixture is filtered. After drying at 80° C. under high vacuum, the crude product is made into slurry with methylene chloride & methanol and filtered to yield Imatinib of the formula (I). Chromatographic separation is used to obtain further crop of product.

After implementing the process described in the patent mentioned as per the scheme indicated, the following are the difficulties encountered and draw backs noticed.
i) column chromatography is necessary to isolate product of formula (I) in pure form and column chromatography technique becomes unpractical on commercial scale.
ii) Usage of the obnoxious and foul smelling chemical pyridine as a solvent and its distillation for work-up makes this process to be abandoned on bulk scale.
iii) Presence of less basic impurities in imatinib base renders it unsuitable for direct conversion of pharmaceutically acceptable salts.

Further in this patent however, the solid state properties of the base are not disclosed. U.S. Pat. No. 6,894,051 describes two crystalline forms of imatinib mesylate, the α-form and the β-form, and WO 2004/106326 describes a crystalline form of imatinib mesylate, designated as form H1, an amorphous imatinib mesylate and crystalline imatinib mesylate hydrate. WO 2005/095379 and WO 2006/024863 describes methods of preparing the imatinib mesylate α-form. US 2006/0223817 describes process for the preparation of crystalline imatinib base, designate as form I

W02005/075454 describes acid addition salts of imatinib such as imatinib tartrate, citrate, malate, fumarate, etc., which are prepared by treatment of imatinib base with the corresponding acid.

One of the important solid state properties of a pharmaceutical substance are its rate of dissolution in aqueous fluid. The rate of dissolution of an active ingredient in a patients stomach fluid may have therapeutic consequences because it imposes an upper limit on the rate at which an orally-administered active ingredient may reach the blood stream.

The solid state form of a compound may also affect its behavior on compaction and its storage stability.

These practical physical characteristics are influenced by the conformation and orientation of molecules in the unit cell, which defines a particular polymorph form of a substance. The polymorphic form may give rise to thermal behavior different form that of the amorphous material (or) another polymorphic form.

Thermal behaviour is measured in the laboratory by such techniques as capillary melting point, Thermo Gravimetric Analysis (TGA) and Differential Scanning Calorimetry (DSC), and may be used to distinguish some polymorphic forms from others. A particular polymorphic form may also give rise to distinct properties that may be detectable by X-Ray Powder Diffraction (XRPD) solid state ¹³CNMR spectrometry and infrared spectrometry.

Various characteristics and properties of the polymorphic forms of a substance. e.g. shape, colour, density and the like, will make one polymorphic form preferable over the others for production and/or pharmaceutical compounding. As a result, a very first step in the processes of product development of a new pharmaceutical agent is the determination of whether it exists in polymorphic forms and if so which of such form possesses advantages for the eventual commercial pharmaceutical application.

Therefore we directed our R & D program to develop an improved process for the preparation of Imatinib base of the formula I and its solid state properties The objective of this study is to provide a new environmentally protective, safe, industrially applicable process, which was devoid of the insufficiencies of the known procedures and makes possible the synthesis of pure compound of the formula I in high yields which is easily realizable industrially.

Accordingly we directed our research based on the points mentioned below
To condense 4-(4-methyl-piperazinomethyl)benzoyl chloride hydrochloride of the formula (III) with N-(5-amino-2-methylphenyl)-4-(3-pyridyl)-2-pyrimidineamine of the formula (II) employing aqueous potassium hydroxide to get imatinib base (scheme-1)

Imatinib base is the precursor of the salt forms of imatinib. As such, there is a need for imatinib base of high purity which may be conveniently used as a precursor in the preparation of highly pure imatinib mesylate or such other salts for therapeutic application. This is especially true when the salts of imatinib base can not be crystallized put from solvents owing to solubility reasons or due to reasons of conversion to undesirable polymorphic forms.

### SUMMARY OF INVENTION

The main object of the present invention is to provide an improved process for the preparation of highly pure (>99.9%) imatinib base

Accordingly in the present invention highly pure Imatinib and its pharmaceutically acceptable salts are prepared by

i. preparing imatinib base by the condensation of N-(5-amino-2-methylphenyl)-4-(3-pyridyl)-2-pyrimidine amine of the formula (II) and 4-(4-methyl-piperazinomethyl)benzoyl chloride of the formula (III) in presence of potassium hydroxide and isolation of imatinib base

ii. Suspension of Imatinib base into purified water and pH adjustment with methane sulphonic acid to 3.0-3.5 and washing with chloroform to remove less basic impurities formed during course of the reaction .

iii. Basification of the aqueous layer to pH 12.0-12.5

iv. Extraction of imatinib base with chloroform

v. Removal of solvent completely under vacuum and precipitating imatinib technical grade product by adding ethyl acetate

vi. Dissolving imatinib technical grade product in chloroform.

vii. Activated carbon treatment

viii. Distillation of Chloroform completely and adding ethyl acetate to afford highly pure imatinib base crystalline form-N of formula (I) of purity>99.9%

### BRIEF DESCRIPTION OF DRAWINGS

FIG. 1 depicts the X-ray powder diffraction pattern of imatinib base crystalline form-N

FIG. 2 depicts the DSC picture of imatinib base crystalline form-N

FIG. 3 depicts the IR spectrum of imatinib base crystalline form-N

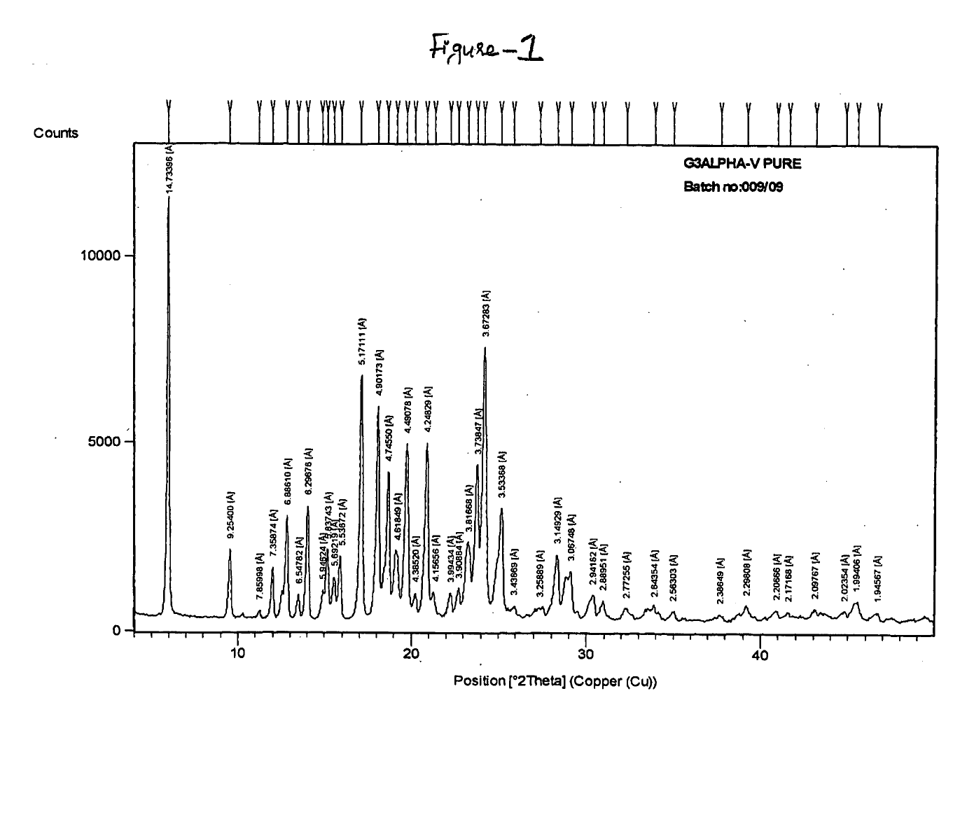

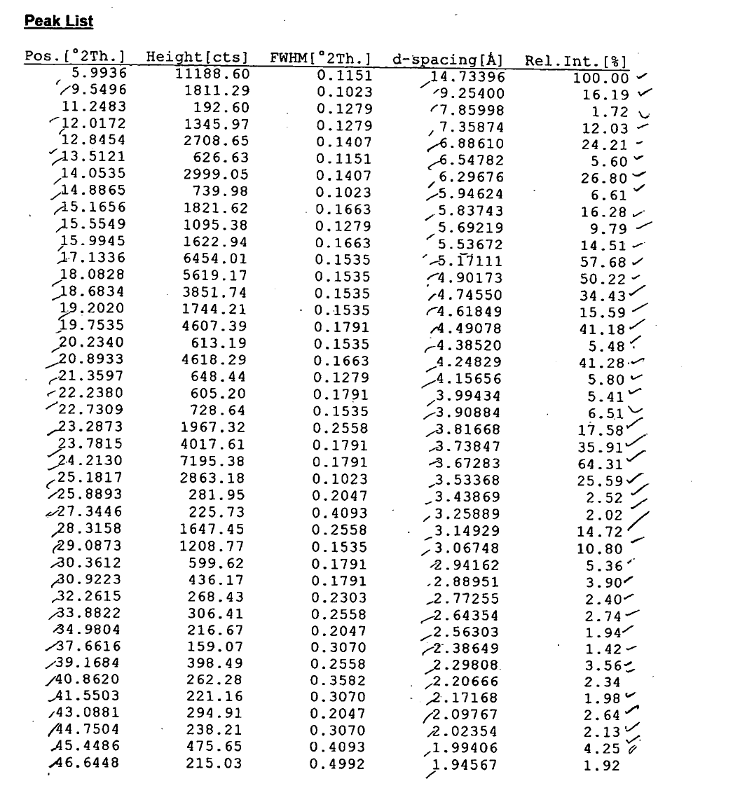

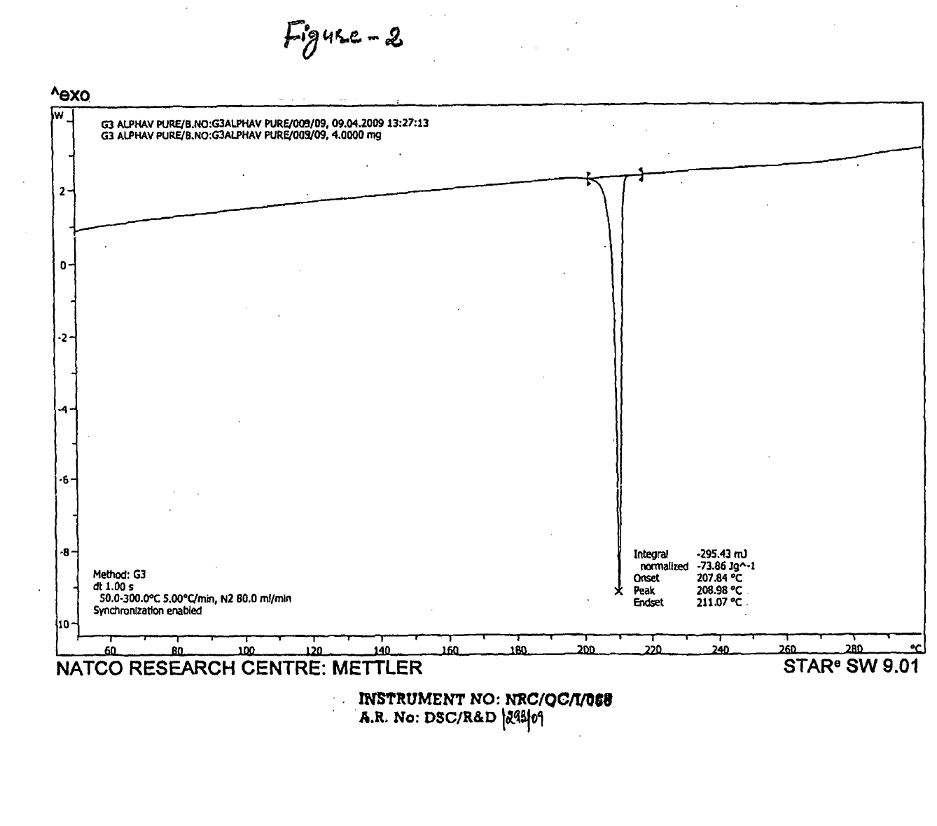

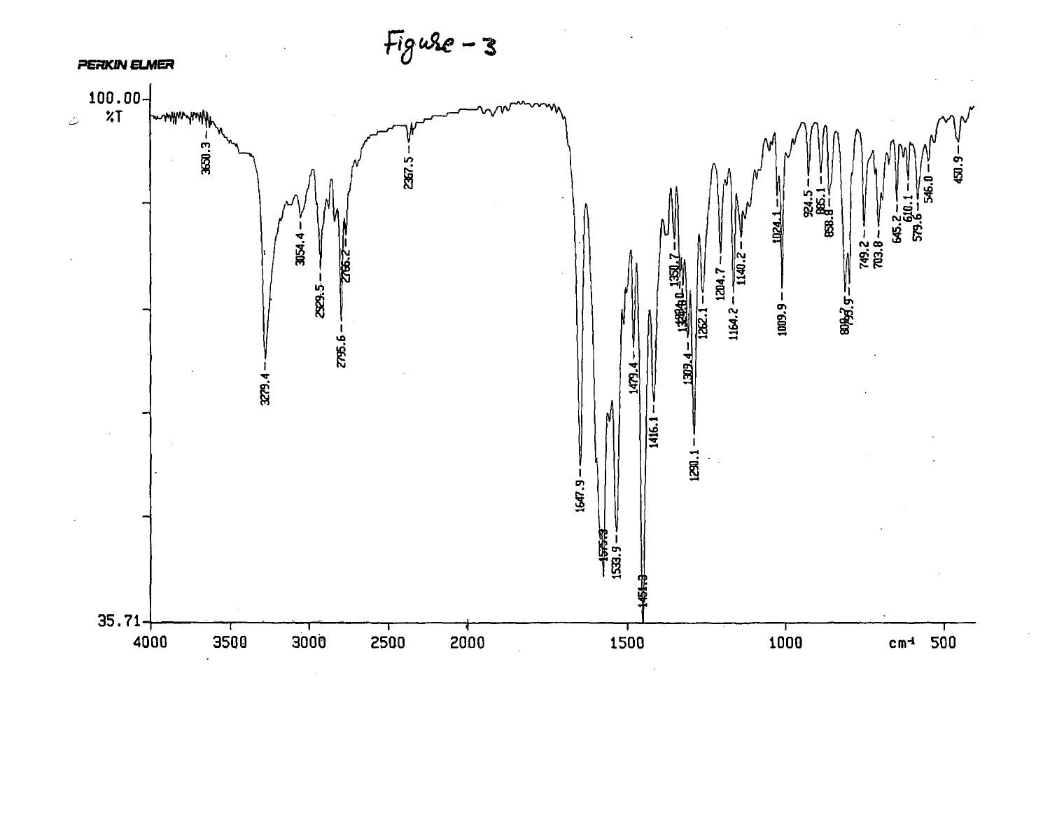

### DETAILED DESCRIPTION OF THE INVENTION

The Imatinib base form-N prepared by the above method produces unique X-ray diffraction pattern as depicted in FIG-1 and Table-1. N-form is characterized by strong diffraction peaks at 5.9, 12.8, 14.0, 17.1, 18.0, 18.7, 19.7, 20.8, 23.8, 24.2, 25.2 +/−0.2 degrees 2θ.

TABLE I
(Imatinib base)

| Angle | d value | Intensity % |
|---|---|---|
| 2-Theta | Angstrom | % |
| 5.9936 | 14.73396 | 100.0 |
| 9.5496 | 9.25400 | 16.19 |
| 11.2483 | 7.85998 | 1.72 |
| 12.0172 | 7.35874 | 12.03 |
| 12.8454 | 6.88610 | 24.21 |
| 13.5121 | 6.54782 | 5.06 |
| 14.0535 | 6.29676 | 26.80 |
| 14.8865 | 5.94624 | 6.61 |
| 15.1656 | 5.83743 | 16.28 |
| 15.5549 | 5.69219 | 9.79 |
| 15.9945 | 5.53672 | 14.51 |
| 17.1336 | 5.17111 | 57.68 |
| 18.0828 | 4.90173 | 50.22 |
| 18.6834 | 4.74550 | 34.43 |
| 19.2020 | 4.61849 | 15.59 |
| 19.7535 | 4.49078 | 41.18 |
| 20.2340 | 4.38520 | 5.48 |
| 20.8933 | 4.24829 | 41.28 |
| 21.3597 | 4.15656 | 5.80 |
| 22.2380 | 3.99434 | 5.41 |
| 22.7309 | 3.90884 | 6.51 |
| 23.2873 | 3.81668 | 17.58 |
| 23.7815 | 3.73847 | 35.91 |
| 24.2130 | 3.67283 | 64.31 |
| 25.1817 | 3.53368 | 25.59 |
| 25.8893 | 3.43869 | 2.52 |
| 27.3446 | 3.25889 | 2.02 |
| 28.3158 | 3.14929 | 14.72 |
| 29.0873 | 3.06748 | 10.80 |
| 30.3612 | 2.94162 | 5.36 |
| 30.9223 | 2.88951 | 3.90 |
| 32.2615 | 2.77255 | 2.40 |
| 33.8822 | 2.64354 | 2.74 |
| 33.8822 | 2.56303 | 1.94 |
| 34.9804 | 2.38649 | 1.42 |
| 37.6616 | 2.29808 | 3.56 |
| 39.1684 | 2.20266 | 2.34 |
| 40.8620 | 2.17168 | 1.98 |
| 41.5503 | 2.09767 | 2.64 |
| 43.0881 | 2.02354 | 2.13 |
| 44.7504 | 1.99406 | 4.25 |
| 45.4486 | 1.94567 | 1.92 |

The Imatinib base form-N prepared by the above method is characterized by IR characteristic absorption peaks 3279.4, 1647, 1575, 1533, 1451, 1479, 1290, 1165, 1010, 926, 810 cm⁻¹ as depicted in FIG-2

Thus in accordance with the present invention preparation of highly pure Imatinib base suitable for conversion to and its pharmaceutically acceptable salts comprise the following steps.

i. Condensation of N-(5-amino-2-methylphenyl)-4-(3-pyridyl)-2-pyrimidine amine of the formula (II) and 4-(4-methyl- piperazinomethyl)benzoyl chloride hydrochloride of the formula (III) in chloroform medium

ii. After reaction completion separation of chloroform layer

iii. Washing of the chloroform layer 5% sodium hydroxide solution and with water successively.

iv. Concentration of the chloroform layer

v. Addition of ethyl acetate to precipitate imatinib base.

vi. Suspension of Imatinib base into purified water and pH adjustment with methane sulphonic acid

vii. extraction with chloroform to remove less basic impurities formed during course of the reaction.

viii. Basification of the aqueous layer with sodium Hydroxide solution.

ix. Extraction of imatinib base with chloroform.

x. Removal of solvent completely under vacuum

xi. Charging ethyl acetate to precipitate imatinib base of technical grade(tech) product

xii. Dissolving imatinib tech into chloroform

xiii. Activated carbon treatment

xiv. Distillation of chloroform completely under vacuum.

xv. Charging ethyl acetate

xvi. Isolation of highly pure imatinib base form-N of formula (I) by filtration.

In a specific embodiment, the present invention provides a process for the preparation of Imatinib which involves:
1. Addition of 30% aqueous solution of potassium hydroxide to a suspension of compound of formulae (II) and (III) in chloroform at 30-35° c
2. after reaction completion separation and storage of aqueous layer to recover 4-(4-methyl piperazino methyl) benzoic acid which the starting material for the preparation of 4-(4-methyl piperazino methyl) benzoyl chloride dihydrochloride of the formula (iii)
3. chloroform layer separation and washing with 5% sodium hydroxide solution and water successively. Distillation of chloroform layer followed by activated carbon treatment
4. filtration of precipitated imatinib base by treating with a mixture of chloroform and ethyl acetate.
5. Suspension of obtained imatinib base in purified water and treatment with methane sulfonic acid to a pH of 3.0-3.5 and washing thoroughly with chloroform to remove less basic impurities
6. aqueous layer pH adjustment with 10% sodium hydroxide solution to 12-12.5 to liberate imatinib base
7. Extraction of aqueous layer with chloroform, separation of chloroform layer and water washing
8. distillation to chloroform and addition of ethyl acetate is added to precipitate imatinib base of technical grade of formula (I)
9. Carbon treatment of imatinib tech in chloroform and distillation of chloroform under vacuum
10. Charging ethyl acetate to precipitate imatinib base form-N
11. Isolation of highly pure imatinib base form-N(>99.8)of formula (I) by filtration.

### STABILITY OF IMATINIB BASE PREPARED BY THE ABOVE PROCESS

1. Pure Imatinib base (1 g) prepared above was sealed in a HDPE bag and kept at 30-35 deg C for three months. XRD analysis indicates that the polymorph-N is stable

Pure imatinib base 1 gm prepared by the process described in Example 1 was taken in a boiling test tube and heated gradually in oil bath the substance was examined by XRD. XRD analysis indicates that polymorph form-N is stable even at elevated temperatures.

The results are tabulated below:

TABLE 2

| | | | | Polymorph form |
|---|---|---|---|---|
| Polymorph content | | Duration of | | detected |
| before heating | Temperature | time | | after heating |
| Imatinib form-N | 110° C. | 2 | h | Form-N |
| Imatinib form-N | 110° C. | 4 | h | Form-N |
| Imatinib form-N | 110° C. | 6 | h | Form-N |
| Imatinib form-N | 110° C. | 8 | h | Form-N |
| Imatinib form-N | 110° C. | 10 | h | Form-N |
| Imatinib form-N | 110° C. | 14 | h | Form-N |
| Imatinib form-N | 30-35° C. | 3 | months | Form-N |

The required N-(5-amino-2-methylphenyl)-4-(3-pyridyl)-2-pyrimidine amine of the formula (II) and 4-(4-methyl-piperazinomethyl)benzoyl chloride dihydrochloride of the formula (III) can be prepared by the prior art processes

The details of the inventions are given in the Examples which are provided for illustration only and therefore the Examples should not be construed to limit the scope of the invention.

### EXAMPLES

### Example-1

### Process for the Preparation of Crystalline Imatinib Base Form-N of the Formula (I)

Condensation of N-(5-amino-2-methylphenyl)-4-(3-pyridyl)-2-pyrimidine amine of the formula (II) and 4-(4-methyl-piperazinomethyl)benzoyl chloride dihydrochloride of the formula (III):

### Raw Materials

| | | |
|---|---|---|
| 1. N-(5-Amino-2-methylphenyl)-4-(3-pyridyl)-2- | 35 | g |
| pyrimidine amine (II) | | |
| 2. 4-(4-Methyl piperazino methyl) benzoyl chloride | 164.1 | g |
| dihydrochloride (III) | | |
| 3. Methane sulfonic acid | 15.2 | g |
| 4. Potassium hydroxide | 340 | g |
| 5. sodium hydroxide flakes | 82 | g |
| 6. Chloroform | 5.0 | L |
| 7. Ethyl acetate | 3.5 | L |
| 8. Activated charcoal | 12 | g |

### Procedure

Step-1: Preparation of imatinib base

N-(5-Amino-2-methylphenyl)-4-(3-pyridyl)-2-pyrimidine amine (II, 35 G) was charged into chloroform (0.7 L) into a 3 L round bottomed flask followed by 4-(4-Methyl piperazino methyl) benzoyl chloride dihydrochloride (III, 164.1 g). Potassium Hydroxide flakes (340 g) were dissolved in 1.1 L DM water to make 30% solution and added this solution to the reaction mass during 4-5 hours at 30-40° C. Chloroform layer was separated and aqueous layer was extracted with chloroform (0.55 L). Chloroform layers were combined and washed with 20% sodium hydroxide solution (prepared by dissolving 72 gms sodium hydroxide flakes in 1.4 L DM water). Chloroform layer was washed thoroughly with DM water. Carbon treatment was given to the chloroform layer at 50-55° C. Chloroform was distilled under vacuum to a residual volume of 240-260 ml. Reaction mass was cooled to room temperature and ethyl acetate (0.85 L) was charged and stirred for 20-30 minutes at room temperature. The product was filtered and washed with Ethyl acetate (100 ml). The filtered base was dried at 50-55° C. Imatinib base yield: 50 g.

Step-2: Preparation of imatinib base technical grade product

Imatinib base (40 g) obtained form step-1 is charged into 1.1 L DM water and stirred for 15-20 minutes. pH was adjusted with Methane sulfonic acid (15.2 g) to 3.0-3.5. Reaction mass was washed with Chloroform (3×275 ml) and aqueous layer PH was adjusted to 12-13 with 10% sodium Hydroxide solution (prepared by dissolving 10 gms in 100 ml). Aqueous layer was extracted twice with Chloroform (1×750 ml, 1×500 ml). Chloroform layer was washed with purified water and distilled under vacuum to a residual volume of 240-260ml. Reaction mass was brought to room temperature and ethyl acetate (1 L) was charged. It was Stirred for 20-30 minutes , filtered and washed with Ethyl acetate (50 ml). Filtered imatinib Tech was dried at 50-55° C. Imatinib Tech yield: 43.5 gms

Step-3: Preparation of imatinib pure base Form-N

Imatinib Tech (43.5 g) from step-2 was charged into Chloroform (1.4 L) and heated to 50-55° C. Carbon treatment was given to the chloroform layer at 50-55° C. Chloroform was distilled under vacuum to a residual volume of 240-260 ml and brought to room temperature. Ethyl acetate (1 L) was charged to the residual chloroform and mass temperature was raised to 50-55° C. Reaction mass was maintained at the same temperature for 30 minutes, filtered at 50-55° C. and washed with Ethyl acetate (100 ml). Filtered imatinib pure base form-N was dried at 50-55° C. Imatinib base (pure) yield: 40 g

Purity by HPLC: 99.9%

XRD: FIG.-1

DSC: FIG.-2

IR: FIG.-3

### ADVANTAGES OF THE INVENTION

1) The Imatinib base is produced in more than 99.8% purity.

2) The process can be used directly for commercial preparation of Imatinib salts of pharmaceutical grade.

3) The process is extremely useful when specific salts of imatinib can not be crystallized for solubility reasons or due to reasons of conversion to undesirable polymorphic forms

4) The process involves separation of less basic impurities present in imatinib base generated by the conventional process thus, purifying the imatinib base

1. Novel process for the preparation of Imatinib base form—N comprising
A. Addition of aqueous solution of potassium hydroxide to a suspension of compounds of formulae (II) and (III) in chloroform at 30-35° C. 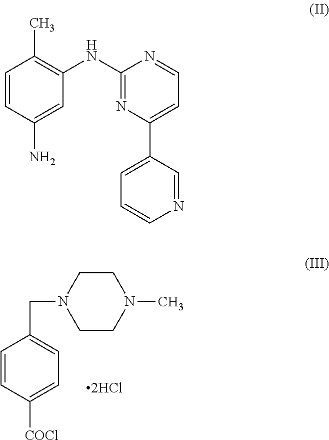 [SMILES: Cc1ccc(N)cc1Nc1nccc(-c2cccnc2)n1 ; CN1CCN(Cc2ccc(C(=O)Cl)cc2)CC1]
B. After reaction completion, chloroform layer separation, washing with sodium hydroxide solution and water successively Distillation of chloroform layer followed by carbon treatment
C. Filtration of imatinib base by treating residue with a mixture of chloroform and ethyl acetate
D. Suspension of obtained imatinib base in demineralised water and treatment with methane sulfonic acid to a pH of 3.0-3.5 and washing thoroughly with chloroform to remove less basic impurities
E. pH adjustment of the separated aqueous layer with 10% sodium hydroxide solution to 12-12.5 to liberate imatinib base
F. Extraction of aqueous layer with chloroform, separation of chloroform layer and water washing
G. Distillation of the chloroform and addition of ethyl acetate to precipitate imatinib base of technical grade
H. Activated carbon treatment of imatinib tech in chloroform and distillation of chloroform under vacuum
I. Charging ethyl acetate and filtration of highly pure imatinib base form-N of formula (I) by filtration (purity>99.8%).

2. (canceled)

3. A method according to claim 1 wherein said aqueous solution of potassium hydroxide in step A is a 30% aqueous solution; said sodium hydroxide solution in step B is a 5% sodium hydroxide solution; and/or said sodium hydroxide solution in step E is a 10% sodium hydroxide solution.
